(19) United States
(12) Patent Application Publication
(10) Pub. No.: US 2004/0028716 A1
(43) Pub. Date: Feb. 12, 2004

(54) Use of Y-27632 as an agent to prevent restenosis after coronary artery angioplasty/stent implantation

(76) Inventors: Andrew R. Marks, Larchmont, NY (US); Steven O. Marx, New York, NY (US)

Correspondence Address: Cooper & Dunham LLP, 1185 Avenue of the Americas, New York, NY, 10036, US

(21) Appl. No.: 10/460,842

(22) Filed: Jun. 12, 2003

Related U.S. Application Data
(60) Provisional application No. 60/388,760, filed on Jun. 14, 2002.
(60) Provisional application No. 60/388,769, filed on Jun. 17, 2002.

(51) Int. Cl. A61K 31/537; A61F 2/00

(52) U.S. Cl. 424/423; 514/238.2

(57) ABSTRACT

This invention is directed to a stent for implantation in a blood vessel, wherein the stent is coated with Y-27632. The invention also provides a method of treating restenosis in a subject which comprises implanting in the subject a stent coated with Y-27632.

This application claims priority of provisional application U.S. Ser. No. 60/388,760, filed Jun. 14, 2002, the contents of which are incorporated herein by reference.

Throughout this application, various publications are referenced in parentheses by author and year. Full citations for these references may be found at the end of the specification immediately preceding the claims. The disclosures of these publications in their entireties are hereby incorporated by reference into this application to more fully describe the state of the art to which this invention pertains.

### BACKGROUND OF THE INVENTION

Coronary artery disease is the leading cause of mortality and morbidity in the developed world. Coronary artery stenting is a leading therapy for coronary artery disease. However, stent restenosis is a major health problem with about 30% incidence (Marx and Marks, 2001). There are currently many strategies being evaluated to prevent coronary artery stent restenosis using compounds on stents. The most promising of these approaches to date is the use of rapamycin (sirolimus)-coated stents (Marx and Marks, 2001; Rensing et al., 2001; Sousa et al., 2001). In clinical studies rapamycin-coated stents have shown a 0% restenosis rate with up to 18 month follow-up. Moreover, the luminal diameter of stented coronary arteries actually increased during follow-up in patients with rapamycin-coated stents. However, prolonged exposure of smooth muscle cells (SMCs) to rapamycin results in development of rapamycin-resistant SMCs (Luo et al., 1996), suggesting that some patients may become resistant to the actions of rapamycin.

The present application discloses the use of Y-27632-coated stents for the prevention of stent restenosis as a novel therapeutic approach that can be used as an alternative to rapamycin-coated stents.

Vascular SMC migration is believed to play a major role in the pathogenesis of many vascular diseases, including restenosis after both percutaneous transluminal angioplasty (PTCA) and coronary stenting (Schwartz, 1997). In normal blood vessels, the majority of SMCs reside in the media or middle coat of the vessel, where they are quiescent and possess a “contractile” phenotype, characterized by the abundance of actin- and myosin-containing filaments. In disease states, SMCs migrate from the media to the intima or inner coat of the blood vessel.

Rapamycin, a macrolide antibiotic, inhibits SMC proliferation both in vitro and in vivo by blocking cell cycle progression at the transition between the first gap (G1) and DNA synthesis (S) phases (Cao et al., 1995; Gallo et al., 1999; Gregory et al., 1993; Marx et al., 1995). The inhibition of cellular proliferation is associated with a marked reduction in cell cycle-dependent kinase activity and in retinoblastoma protein phosphorylation in vitro (Marx et al., 1995) and in vivo (Gallo et al., 1999). Down-regulation of the cyclin-dependent kinase inhibitor (CDKI) p27^{kip1} by mitogens is blocked by rapamycin (Kato et al., 1994; Nourse et al., 1994). In p27^{kip1} (−/−) knockout mice, relative rapamycin resistance was demonstrated, and in rapamycin-resistant myogenic cells, constitutively low levels of p27^{kip1} were observed, which were not increased by serum withdrawal or the addition of rapamycin (Luo et al., 1996). It has been shown that rapamycin inhibits rat, porcine, and human SMC migration (Poon et al., 1996). It has been shown further that rapamycin has potent inhibitory effects on SMC migration in wild type and p27 (+/−) mice, but not in p27 (−/−) knockout mice, indicating that the cyclin-dependent kinase inhibitor (CDKI) p27^{kip1} plays a critical role in rapamycin's anti-migratory properties and in the signaling pathway(s) that regulates SMC migration (Sun et al., 2001).

C3 exoenzyme inhibits thrombin-mediated vascular SMC proliferation and migration (Seasholtz et al., 1999). Like rapamycin, C3 exoenzyme inhibits vascular SMC migration in wild type and in p27 null mice, indicating that C3 exoenzyme acts via both p27-dependent and p27-independent pathways (see FIG. 1) (Sun et al., 2001). C3 exoenzyme inhibits vascular SMC migration and proliferation in part by inhibiting RhoA which is involved in regulating p27 degradation.

Y-27632, relative molecular mass 338.3, is a potent inhibitor of Rho-kinase. Agents that inhibit Rho-kinase potently inhibit both proliferation and migration of SMCs through a p27kip¹-independent and p27kip¹-dependent mechanism (FIGS. 1 and 2) (Seasholtz et al., 1999; Sun et al., 2001).

### SUMMARY OF THE INVENTION

The present invention is directed to a stent for implantation in a blood vessel, wherein the stent is coated with Y-27632.

This invention provides a method for treating or preventing restenosis in a subject which comprises implanting in the subject a stent coated with Y-27632.

This invention also provides a stent for implantation in a blood vessel, wherein the stent is coated with an inhibitor of RhoA.

This invention further provides a method for treating or preventing restenosis in a subject which comprises implanting in the subject a stent coated with an inhibitor of RhoA.

In addition, the present invention provides a stent for implantation in a blood vessel, wherein the stent is coated with an inhibitor of Rho kinase.

Finally, this invention also provides a method for treating or preventing restenosis in a subject which comprises implanting in the subject a stent coated with an inhibitor of Rho kinase.

### BRIEF DESCRIPTION OF THE FIGURES

FIG. 1. Rapamycin and C3 exoenzyme inhibit SMC migration through p27^{kip1}-dependent and p27^{kip1}-independent pathways. Growth factor receptor activation by mitogens/nutrients activate PI3-kinase, which indirectly (dashed lines) stimulates mTOR, p70^{s6k} and RhoA. Rapamycin (RAPA)-FKBP12 inhibits TOR-mediated activation/phosphorylation of protein translation modulators (p70^{s6k}) and prevents mitogen-induced down-regulation of p₂₇^{kip1} through an unknown mechanism (lines with bars indicate inhibitory effects; arrows indicate stimulatory effects). Rapamycin inhibits SMC migration through both p27^{kip1}-dependent and p₂₇^{kip1}-independent mechanisms. C3 exoenzyme, which specifically ADP-ribosylates and inhibits RhoA, inhibits SMC migration through p27^{kip1}-dependent and p27^{kip1}-independent (cytoskeleton effects) pathways (Sun et al., 2001).

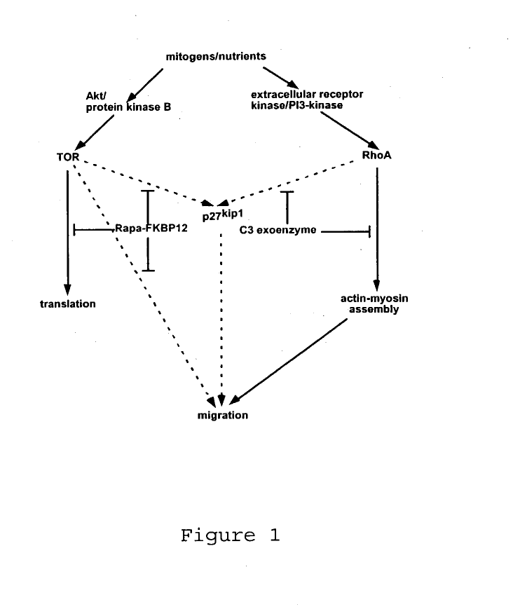

FIG. 2. RhoA and ROCK (Rho kinase) modulate cell cycle regulators, calcium sensitivity and migration/cytokinesis. C3 exoenzyme and Y-27632 inhibit mitogen-induced proliferation and migration through inhibition of RhoA and ROCK respectively.

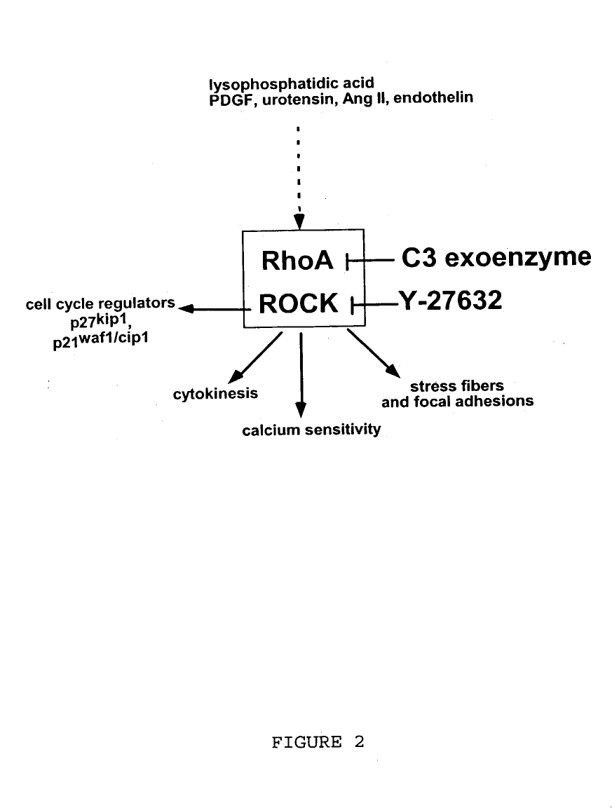

### DETAILED DESCRIPTION OF THE INVENTION

The present invention is directed to a stent for implantation in a blood vessel, wherein the stent is coated with Y-27632. Y-27632 (Cat. No. 688000, Calbiochem-Novabiochem Corp.), which has a relative molecular mass of 338.3, is a potent inhibitor of Rho-kinase. Y-27632 has the structure:

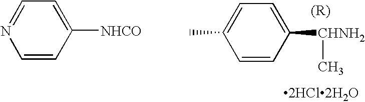

This invention is directed to a stent for implantation in a blood vessel, wherein the stent is coated with a compound having the structure:

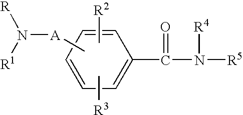

wherein

R is a hydrogen, an alkyl, or a cycloalkyl, a cycloalkylalkyl, a phenyl or an aralkyl, which optionally has a substituent on a ring, or a group of the formula

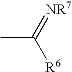

wherein R⁶ is hydrogen, alkyl or the formula: —NR⁸R⁹ wherein R⁸ and R⁹ are the same or different and each is hydrogen, alkyl, aralkyl or phenyl, and

R⁷ is hydrogen, alkyl, aralkyl, phenyl, nitro or cyano, or

R⁶ and R⁷ combinedly form a heterocycle optionally having oxygen atom, sulfur atom or optionally substituted nitrogen atom additionally in the ring;

R¹ is a hydrogen, an alkyl, or a cycloalkyl, a cycloalkylalkyl, a phenyl or an aralkyl, which optionally has a substituent on a ring; or

R and R¹ combinedly form, together with the adjacent nitrogen atom, a heterocycle optionally having oxygen atom, sulfur atom or optionally substituted nitrogen atom additionally in the ring;

R² and R³ are the same or different and each is a hydrogen, an alkyl, an aralkyl, a halogen, a nitro, an amino, an alkylamino, an acylamino, a hydroxy, an alkoxy, an aralkyloxy, a cyano, an acyl, a mercapto, an alkylthio, an aralkylthio, a carboxy, an alkoxycarbonyl, a carbamoyl, an alkylcarbamoyl or an azide;

R⁴ is a hydrogen or an alkyl;

R⁵ is a heterocycle containing nitrogen, which is selected from the group consisting of pyridine, pyrimidine, pyridazine, triazine, pyrazole, triazole, pyrrolopyridine, pyrazolopyridine, imidazopyridine, pyrrolopyrimidine, pyrazolopyrimidine, imidazopyrimidine, pyrrolotriazine, pyrazolotriazine, triazolopyridine, triazolopyrimidine, cinnoline, quinazoline, quinoline, pyridopyridazine, pyridopyrazine, pyridopyrimidine, pyrimidopyrimidine, pyrazinopyrimidine, naphthylidine, tetrazolopyrimidine, thienopyridine, thienopyrimidine, thiazolopyridine, thiazolopyrimidine, oxazolopyridine, oxazolopyrimidine, furopyridine, furopyrimidine, 2,3-dihydropyrrolopyridine, 2,3-dihydropyrrolopyrimidine, 5,6,7,8-tetrahydropyrido-[2,3-d]pyrimidine, 5,6,7,8-tetrahydro-1,8-naphthylidine and 5,6,7,8-tetrahydroquinoline, provided that when said heterocycle containing nitrogen forms a hydrogenated aromatic ring, carbon atom in the ring is optionally carbonyl, and said heterocycle containing nitrogen optionally has a substituent; and

A is the formula

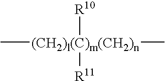

wherein R¹⁰ and R¹¹ are the same or different and each is hydrogen, alkyl, haloalkyl, aralkyl, hydroxyalkyl, carboxy or alkoxycarbonyl, or R¹⁰ and R¹¹ combinedly form cycloalkyl, and, m and n are each 0 or an integer of 1-3,

or an isomer thereof.

This compound is described in U.S. Pat. Nos. 5,958,944 and 6,156,766.

The present invention is also directed to a stent for implantation in a blood vessel, wherein the stent is coated with a compound having the structure:

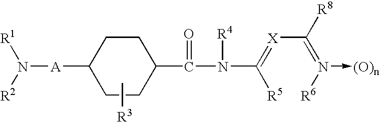

wherein:

R¹ and R² are the same or different and each is hydrogen, alkyl, or cycloalkyl, cycloalkylalkyl, phenyl, aralkyl, piperidyl or pyrrolidinyl, which may have substituent on the ring, or a group of the formula

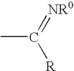

wherein

R is hydrogen, alkyl, —NR′R″ (where R′ and R″ are the same or different and each is hydrogen, alkyl, aralkyl or phenyl),

R⁰ is hydrogen, alkyl, aralkyl, phenyl, nitro or cyano, or

R and R⁰ may combinedly form a heterocyclic ring which may have, in the ring, oxygen atom, sulfur atom or optionally substituted nitrogen atom, or

R¹ and R² combinedly are alkylidene or phenylalkylidene, or

R¹ and R² form, together with the nitrogen atom binding therewith, a heterocyclic ring which may have, in the ring, oxygen atom, sulfur atom or optionally substituted nitrogen atom;

R³ and R⁴ are each hydrogen or alkyl;

A is a single bond or alkylene;

X is ═C(R⁷)— or ═N—;

R⁵ and R⁶ together are a group of the formula

—CRa═CRb—,

—NRa—C(═Rb)— or

—C(═Ra)—NRb—,

wherein

Ra and Rb combinedly form an optionally hydrogenated 5- or 6-membered aromatic ring which may have, in the ring, at least one of nitrogen atom, sulfur atom and oxygen atom;

R⁷ and R⁸ are the same or different and each is hydrogen, halogen, alkyl, alkoxy, aralkyl, haloalkyl, nitro, —NReRf {wherein Re and Rf are the same or different and each is hydrogen, alkyl, —COR⁹, —COOR^{9′}, —SO₂R⁹ (where R^{9′} is hydrogen, alkyl, phenyl or aralkyl and R^{9′} is alkyl, phenyl or aralkyl), or Re and Rf form, together with the nitrogen atom binding therewith, a heterocyclic ring which may have, in the ring, oxygen atom, sulfur atom or optionally substituted nitrogen atom}, cyano, azido, optionally substituted hydrazino, —COOR¹⁰, —CONR¹¹R¹² (wherein R¹⁰⁻¹² are each hydrogen, alkyl, phenyl or aralkyl); and

n is 0 or 1;

or an isomer thereof.

This compound is described in U.S. Pat. No. 5,478,838.

This invention provides a stent for implantation in a blood vessel, wherein the stent is coated with a compound having the structure:

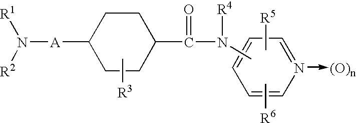

wherein

R¹ and R² are the same or different, and respectively represent:

hydrogen, C₁₋₁₀ alkyl, C₂₋₅ alkanoyl, formyl, C₁₋₄ alkoxycarbonyl, amidino, C₃₋₇ cycloalkyl, C₃₋₇ cycloalkyl-carbonyl, unsubstituted or substituted phenyl, phenylalkyl, benzoyl, naphthoyl, phenylalkoxy-carbonyl, pyridylcarbonyl or piperidyl, wherein the substituent is selected from the group consisting of halogen, C₁₋₄ alkyl, C₁₋₄ alkoxy, phenylalkyl, nitro or amino,

R¹ and R² together form unsubstituted or substituted benzylidene, pyrrolidylidene or piperidylidene, wherein the substituent is selected from the group consisting of halogen, C₁₋₄ alkyl, C₁₋₄ alkoxy, phenylalkyl, nitro or amino, or

R¹ or R² together with the adjacent nitrogen atom form pyrrolidinyl, piperidino, piperazinyl, morpholino, thiomorpholino or phthalimido,

R³ represents hydrogen or C₁₋₄ alkyl,

R⁴ represents a hydrogen or C₁₋₄ alkyl,

R⁵ represents hydrogen, hydroxy, C₁₋₄ alkyl or phenylalkoxy,

R⁶ represents hydrogen or C₁₋₄ alkyl,

A represents single bond, C₁₋₅ straight chain alkylene, or alkylene which is substituted by C₁₋₄ alkyl and

n represents 0 to 1,

or an isomer thereof.

This compound is described in U.S. Pat. No. 4,997,834.

This invention also provides a stent for implantation in a blood vessel, wherein the stent is coated with a compound comprising an amide compound having the structure:

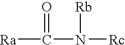

wherein

Ra is a group of the formula:

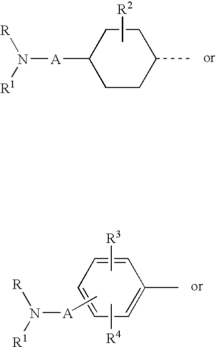

in the formulas (a) and (b),

R is hydrogen, alkyl or cycloalkyl, cycloaalkyl, phenyl or aracyl, which optionally have a substituent on the ring, or a group of the formula:

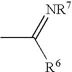

wherein R⁶ is hydrogen, alkyl or formula: —NR⁸NR⁹ wherein R⁸ and R⁹ are the same or different and each is hydrogen, alkyl, aralkyl or phenyl, R⁷ is hydrogen, alkyl, aralkyl, phenyl, nitro or cyano, or R⁶ and R⁷ in combination show a group forming a heterocycle optionally having, in the ring, oxygen atom, sulfur atom or optionally substituted nitrogen atom,

R¹ is hydrogen, alkyl or cycloalkyl, cycloalkylalkyl, phenyl or aralky, which optionally have a substituent on the ring, or

R and R¹ in combination form, together with the adjacent nitrogen atom, a group forming a heterocycle optionally having, in the ring, oxygen atom, sulfur atom or optionally substituted nitrogen atom,

R² is hydrogen or alkyl,

R³ and R⁴ are the same or different and each is hydrogen, alkyl, aralkyl, halogen, nitro, amino, alkylamino, acylamino, hydroxy, alkoxy, aralkyloxy, cyano, acyl, mercapto, alkylthio, aralkylthio, carboxy, alkoxycarbonyl, carbamoyl, alkylcarbamoyl or azide, and

A is a group of the formula:

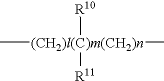

wherein R¹⁰ and R¹¹ are the same or different and each is hydrogen, alkyl, haloalkyl, aralkyl, hydroxyalkyl, carboxy or alkoxycarbonyl, or R¹⁰ and R¹¹ show a group which forms cycloalkyl in combination and l, m and n are each 0 or an integer of 1-3,

Rb is a hydrogen, an alkyl, an aralkyl, an aminoalkyl or a mono or dialkylaminoalkyl; and

Rc is an optionally substituted pyridine, triazine, pyrimidine, pyrrolopyridine, pyrazolopyridine, pyrazolopyrimidine, 2,3-dihydropyrrolopyridine, imidazopyridine, pyrrolopyrimidine, imindazopyrimidine, pyrrolotriazine, pyrazolotriazine, triazolopyridine, triazolopyrimidine, or 2,3-dihydropyrrolopyrimidine,

or an isomer thereof.

This compound is described in U.S. Pat. No. 6,218,410 B1.

This invention further provides a stent for implantation in a blood vessel, wherein the stent is coated with a compound having the structure:

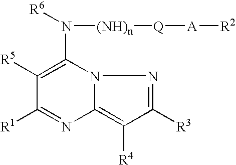

wherein

R¹ is hydrogen, lower alkyl which may have thienyl, lower alkoxy, lower alkylthio, oxo or hydroxyl as a substituent, cycloalkyl, thienyl, furyl, lower alkenyl, or R¹ phenyl, said R¹ phenyl having 1 to 3 substituents selected from the group consisting of lower alkyl, lower alkoxy, phenylthio and halogen; R² is naphthyl, cycloalkyl, furyl, thienyl, pyridyl, halogen-substituted pyridyl, phenoxy, halogen-substituted phenoxy, or phenyl which may have 1 to 3 substituents selected from the group consisting of lower alkyl, lower alkoxy, halogen, nitro, halogen-substituted lower alkyl, halogen-substituted lower alkoxy, lower alkoxycarbonyl, hydroxyl, phenyl(lower)alkoxy, amino, cyano, lower alkanoyloxy, phenyl and di(lower)alkoxyphosphoryl(lower)alkyl; R³ is hydrogen, phenyl or lower alkyl; R⁴ is hydrogen, lower alkyl, lower alkoxycarbonyl, phenyl(lower)alkyl, phenyl, phenylthio-substituted phenyl, or halogen; R⁵ is hydrogen or lower alkyl; R⁶ is hydrogen, lower alkyl, phenyl(lower)alkyl, or an R⁶ benzoyl, said R⁶ benzoyl having 1 to 3 substituents selected from the group consisting of lower alkoxy, halogen-substituted lower alkyl and halogen; R¹ and R⁵ may conjointly form lower alkylene; Q is carbonyl or sulfonyl; A is a single bond, lower alkylene or lower alkenylene; and n is 0 or 1,

or an isomer thereof.

This compound is described in U.S. Pat. No. 5,707,997.

In addition, the present invention provides a stent for implantation in a blood vessel, wherein the stent is coated with an inhibitor of Rho kinase. In different embodiments the inhibitor of Rho kinase is Y-27623, Y-30141, Y-33075, Y-32885, Y-30964, Y-28791, HA1077 (fasudil), hydroxyfasudil, or H-7 (U.S. Pat. No. 6,218,410 B1; Uehata et al., 1997).

The invention is also directed to a stent for implantation in a blood vessel, wherein the stent is coated with an inhibitor of RhoA. In one embodiment, the inhibitor of RhoA is C3 exoenzyme. In another embodiment, the C3 exoenzyme is botulinum toxin C3 exoenzyme. In still another embodiment, toxin A and/or toxin B from C. difficle that has similar effects as C3 exoenzyme (Muniyappa et al., 2000) is used. In one embodiment, the inhibitor of RhoA is a HMG CoA reductase inhibitor. In another embodiment the inhibitor is a statin. In different embodiments the statin is one or more of lovastatin, simvastatin, pravastatin, fluvastatin, atorvastatin, or cerivastatin. In one embodiment, the inhibitor of RhoA is a geranylgeranyl transferase inhibitor. In another embodiment, the inhibitor is GGTI-298 (Lerner et al., 1995). In a further embodiment, the inhibitor inhibits prenylation of RhoA, thereby inhibiting its function.

This invention also provides a stent for implantation in a blood vessel, wherein the stent is coated with an agent that elevates levels of cyclin-dependent kinase inhibitor p27.

In one embodiment of any of the stents described herein, the stent is also coated with rapamycin. In another embodiment, the stent is also coated with taxol. In a further embodiment, the stent is also coated with actinomycin D. In a still further embodiment, the stent is also coated with two or more of rapamycin, taxol, or actinomycin D. In yet another embodiment, the stent is also coated with heparin.

This invention is also directed to a stent for implantation in a blood vessel, wherein the stent is coated with an adenoviral vector (see, e.g., U.S. Pat. No. 6,290,949 B1). In one embodiment, the adenovirus expresses dominant negative Rho kinase (Eto et al., 2000). In another embodiment, the adenovirus is used to deliver C3 exoenzyme.

In different embodiments, the stent can be coated with different combinations of any of the agents described herein.

In different embodiments, the compounds or agents are released at different rates from the stent.

C3 exoenzyme enters cells passively with prolonged exposure (e.g., 24-49 hours) which would be afforded with a formulation that elutes off from stents over days as has been developed for rapamycin. Different formulations can provide different rates of release of the drugs from the stent.

Chimeric molecules in which the active site of C3 exoenzyme or other agents is fused to regions of toxins that are rapidly taken up into cells can be generated to enhance the uptake of C3 exoenzyme or the agent into cells. Similarly, viral agents can be used to enhance entry of C3 or other agents on the stent into cells. Other ways of enhancing entry of C3 or an agent into a cell include, but are not limited to, combining C3 or the agent with any of the following: a peptide added with C3 exoenzyme or the agent, a leader sequence comprising an amino acid sequence (e.g., 9 arginines or 9 lysines or combinations thereof) fused to C3 exoenzyme or to the agent, or a TAT sequence based upon the HIV-1 viral sequence.

This invention also provides stents coated with homologs, analogs, isomers, isoforms, or isozymes of any of the compounds or agents described herein, and the use of such stents in any of the methods described herein. A structural and functional analog of a chemical compound has a structure similar to that of the compound but differing from it in respect to a certain component or components. A structural and functional homolog of a chemical compound is one of a series of compounds each of which is formed from the one before it by the addition of a constant element. The term “analog” is broader than and encompasses the term “homolog.” Isomers are chemical compounds that have the same molecular formula but different molecular structures or different arrangement of atoms is space. The isomers may be structural isomers, positional isomers, stereoisomers, optical isomers, or cis-trans isomers. The invention also provides for keto-enol tautomers. Isoforms are multiple forms of a protein whose amino acid sequences differ slightly but whose general activity is identical. Isozymes (isoenzymes) are multiple forms of an enzyme that catalyze the same reaction but differ from each other in properties such as substrate affinity or maximum rate of enzyme-substrate reaction.

This invention also provides stents coated with prodrugs or metabolites of any of the compounds or agents described herein, and the use of such stents in any of the methods described herein. In general, prodrugs will be functional derivatives of compounds which are readily convertible in vivo into the required compound. Conventional procedures for the selection and preparation of suitable prodrug derivatives are described, for example, in Design of Prodrugs, ed. H. Bundgaard, Elsevier, 1985. Metabolites include active species produced upon introduction of compounds into the biological milieu.

This invention also provides intravascular devices other than stents which are coated with any of the compounds or agents described herein.

The present invention provides for the use of any of the stents disclosed herein for prevention or treatment of restenosis. In one embodiment, the restenosis occurs after angioplasty. In another embodiment, the restenosis occurs after vascular stent placement. In different embodiments, the restenosis occurs after coronary artery stent placement, peripheral artery stent placement, or cerebral artery stent placement. In other embodiments, the stent is implanted in a coronary artery, a peripheral artery, a cerebral artery, or a vascular shunt including arterio-venous shunts used for kidney dialysis.

This invention also provides a method of treating restenosis in a subject which comprises implanting in the subject any one of the stents disclosed herein. As used herein, “subject” means any animal, such as a mammal or a bird, including, without limitation, a cow, a horse, a sheep, a pig, a dog, a cat, a rodent such as a mouse or rat, a turkey, a chicken and a primate. In the preferred embodiment, the subject is a human being.

This invention further provides a method of preventing restenosis in a subject which comprises implanting in the subject any one of the stents disclosed herein. In one embodiment, the restenosis occurs after angioplasty. In another embodiment, the restenosis occurs after vascular stent placement. In different embodiments, the restenosis occurs after coronary artery stent placement, peripheral artery stent placement, or cerebral artery stent placement. In other embodiments, the stent is implanted in a coronary artery, a peripheral artery, or a cerebral artery.

The present invention still further provides a method of preventing or treating a condition in a subject which comprises implanting in the subject any one of the stents or intravascular devices disclosed herein. In different embodiments, the condition is peripheral vascular disease, neurovascular disease, platelet aggregation, T cell activation and/or proliferation, or vasospasm including Prinzmetal's angina, migraine headaches or vascular headaches. In one embodiment, the agent released from the stent is used to cause local vasorelaxation of the vessel wall. This may be used to treat vasospasm, e.g. after balloon injury.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

Expression of C3 Exoenzyme

C3 exoenzyme can be prepared as previously described (Dillon and Feig, 1995). Competent cells of Escherichia coli strain BL21 were transformed with a glutathione-S-transferase (GST)-C3 exoenzyme cDNA (gift of Dr. Judy Meinkoth, University of Pennsylvania). Protein expression was induced with 200 μM isopropylthiogalactoside (IPTG) at 32° C. for 3 hours. Lysates were prepared and incubated with GST-Sepharose beads for 1 hour at 4° C. The beads were washed and incubated overnight at 4° C. with 3 units/ml of thrombin (for cleavage of the C3 exoenzyme from the GST fusion protein), which was removed by incubating the supernatant with antithrombin-Sepharose beads for 1 hour at 4° C. The supernatant was concentrated with a Centricon-10 (Amicon Inc., Beverly, Mass). Protein concentration was determined by Bradford assay and the supernatant was aliquoted and frozen in liquid nitrogen. The samples were subjected to SDS-polyacrylamide gel electrophoresis (SDS-PAGE) and stained with Coomassie Blue to confirm correct expression of the GST fusion protein and cleavage/purification of C3 exoenzyme before use (Seasholtz et al., 1999).

C3 exoenzyme is commercially available from Biomol Research Laboratories and List Biological Laboratories.

Drug Incorporation in Stents

Along with an increase in the use of stenting to treat coronary artery disease, there has been a significant increase in attempts to incorporate drugs in stents, with the objective of making these drugs available locally to the vessel wall as an anti-restenotic therapeutic. The aim has been to incorporate the therapeutic agent in the stent such that the rate of drug elution from the stent, and the duration for which this elution continues, is controlled in a pre-determined and reproducible manner.

The ways in which therapeutic agents have been incorporated into stents for uptake by the vessel wall can be broadly classified into two categories: 1) using a polymer to formulate, coat and release the therapeutic agent; and 2) incorporating the agent directly onto the metallic stent by suitably modifying the stent, e.g., by introducing pores or other reservoir systems for holding the agent, with the use of a suitable release mechanism, such as the use of membranes.

The first category of using polymers to incorporate the drug, which has been the more widely attempted method, can be further divided into two classes: 1) use of polymers which are “permanent'” i.e., which remain on the stent after the drug elution from the stent has stopped; and 2) use of polymers which are degradable or erodible in the vasculature, and are completely expended as the drug elution is complete. In the former case where the polymer(s) remains after the drug has eluted out, diffusion of the drug through and out of the polymer is the controlling mechanism for the rate and duration of drug elution. In the case of degradable polymers, the release of the drug proceeds in conjunction with the degradation of the polymer, which typically becomes the controlling mechanism.

When a polymer is used, a solvent is typically used to blend and formulate the polymer and therapeutic agent, and the mixture is coated onto the metallic stent by dip coating, spray coating or other means. On drying, the polymer-drug mixture remains on the stent. The criteria for the suitable selection of the polymer(s) for the particular drug include ability to achieve controlled delivery of the drug at a desired rate for a desired duration, biocompatibility, mechanical integrity during stent expansion and post implant in a pulsatile flow environment.

The following are some of the ways in which therapeutic agents have been incorporated onto stents.

Chudzik et al. (U.S. Pat. No. 6,344,035) have used a mixture of two polymers (polybutyl methacrylate and polyethylene co-vinyl acetate), by varying the compositions of which, rates and durations of drug elution can be controlled. These are permanent polymers.

Yang et al. (U.S. Pat. No. 6,258,121) used a mixture of two coatings, a hydrophilic polylactic acid-polyethylene oxide and a hydrophobic coating of polylactic acid-polycaprolactone to hold and release Taxol. This is an example of degradable coating.

Guruwaiya et al. (U.S. Pat. No. 6,251,136) used a sticky substance (fibronectin, gelatin, collagen) as a base layer, on which a therapeutic agent is sprayed as a dry, micronized powder, with a polymeric cover of ethylene vinyl alcohol acting as the rate controlling mechanism. This is an example of use of polymers to control the diffusion rate, but not the formulation of the agent.

Vectoris Corporation has developed polyester-type polymers using alpha amino acids and PCEL types of polymers from L-lactide, caprolactone and polyethylene glycol monomers. These are biodegradable stent coatings, with the drugs being attached covalently to the polymers.

Wright et al. (U.S. Pat. No. 6,273,913) introduced micropores in the stent body to load and deliver Rapamycin.

Vascular stents are commercially available from Cordis Co., Warren, N.J. Stent implantation procedures are well known in the art (see, e.g., Sousa et al., 2001).

### REFERENCES

Bundgaard, H. (ed.) (1985) Design of Prodrugs, Elsevier, 1985.

Cao, W., Mohacsi, P., Shorthouse, R., Pratt R. and Morris, R. (1995) Effects of rapamycin on growth factor-stimulated vascular smooth muscle cell DNA synthesis: inhibition of basic fibroblast growth factor and platelet-derived growth factor action and antagonism of rapamycin by FK506. Transplantation 59: 390-395.

Dillon, S. T. and Feig, L. A. (1995) Purification and assay of recombinant C3 transferase. Methods in Enzymology 256: 174-184.

Eto, Y. et al. (2000) Gene transfer of dominant negative Rho kinase suppresses neointimal formation after balloon injury in pigs. Am J Heart Circ Physiol 278(6): H1744-1750.

Gallo, R. et al. (1999) Inhibition of intimal thickening after balloon angioplasty in porcine coronary arteries by targeting regulators of the cell cycle. Circulation 99: 2164-2170.

Gregory, C., Huie, P., Billingham, M. and Morris, R. (1993) Rapamycin inhibits arterial intimal thickening caused by both alloimmune and mechanical injury. Transplantation 55: 1409-1418.

Kato, J. M., Matsuoka, M., Polyak, K., Massague, J. and Sherr, C. J. (1994) Cyclic AMP-induced G1 phase arrest mediated by an inhibitor (p27kip1) of cyclin-dependent kinase-4 activation. Cell 79: 487-496.

Lerner et al. (1995) Disruption of oncogenic K-Ras4B processing and signaling by a potent geranylgeranyltransferase I inhibitor. J. Biol. Chem. 270: 26770-26773.

Luo, Y., Marx, S. O., Kiyokawa, H., Koff, A., Massague, J. and Marks, A. R. (1996) Rapamycin resistance tied to defective regulation of p₂₇^{kip1}. Mol. Cell. Biol. 16: 6744-6751.

Marx, S. O., Jayaraman, T., Go, L. O. and Marks, A. R. (1995) Rapamycin-FKBP inhibits cell cycle regulators of proliferation in vascular smooth muscle cells. Circ Res 76: 412-417.

Marx, S. O. and Marks, A. R. (2001) Bench to bedside: The development of rapamycin and its application to stent restenosis. Circulation 104: 852-855.

Muniyappa, R. et al. (2000) Inhibition of Rho protein stimulates iNOS expression in rat vascular smooth muscle cells. Am J Heart Circ Physiol. 278: H1762-1768.

Nourse, J. et al. (1994) Interleukin-2-mediated elimination of the p27kip1 cyclin-dependent kinase inhibitor prevented by rapamycin. Nature 372: 570-573.

Poon, M., Marx, S. O., Gallo, R., Badimon, J. J., Taubman, M. B. and Marks, A. R. (1996) Rapamycin inhibits vascular smooth muscle cell migration. J. Clin. Invest. 98: 2277-2283.

Rensing, B. J. et al. (2001) Coronary restenosis elimination with a sirolimus eluting stent. European Heart Journal 22: 2125-2130.

Schwartz, S. M. (1997) Smooth muscle migration in atherosclerosis and restenosis. J. Clin. Invest. 100: S87-98.

Seasholtz, T. M. et al. (1999) Rho and Rho kinase mediate thrombin-stimulated vascular smooth muscle cell DNA synthesis and migration. Circ Res 84: 1186-1193.

Sousa, J. E. et al. (2000) Lack of neointimal proliferation after implantation of sirolimus-coated stents in human coronary arteries. A quantitative coronary angiography and three-dimensional intravascular ultrasound study. Circulation 102: r54-r57.

Sun, J. et al. (2001) Role for p27(Kip1) in vascular smooth muscle cell migration. Circulation 103(24): 2967-2972.

Uehata, M. et al. (1997) Calcium sensitization of smooth muscle mediated by a Rho-associated protein kinase in hypertension. Nature 389: 990-994.

U.S. Pat. No. 6,344,035, issued Feb. 5, 2002, Chudzik et al.

U.S. Pat. No. 6,258,121, issued Jul. 10, 2001, Yang et al.

U.S. Pat. No. 6,251,136, issued Jun. 26, 2001, Guruwaiya et al.

U.S. Pat. No. 6,273,913, issued Aug. 14, 2001, Wright et al.

U.S. Pat. No. 5,958,944, issued Sep. 28, 1999, Arita et al.

U.S. Pat. No. 6,156,766, issued Dec. 5, 2000, Arita et al.

U.S. Pat. No. 5,478,838, issued Dec. 26, 1995, Arita et al.

U.S. Pat. No. 4,997,834, issued Mar. 5, 1991, Muro et al.

U.S. Pat. No. 5,707,997, issued Jan. 13, 1998, Shoji et al.

U.S. Pat. No. 6,218,410 B1, issued Apr. 17, 2001, Uehata et al.

U.S. Pat. No. 6,290,949 B1, issued Sep. 18, 2000, French et al.

What is claimed is:

1. A stent for implantation in a blood vessel, wherein the stent is coated with Y-27632.

2. A stent for implantation in a blood vessel, wherein the stent is coated with a compound having the structure: 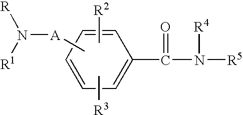
wherein
R is a hydrogen, an alkyl, or a cycloalkyl, a cycloalkylalkyl, a phenyl or an aralkyl, which optionally has a substituent on a ring, or a group of the formula 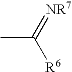
wherein R⁶ is hydrogen, alkyl or the formula: —NR⁸R⁹ wherein R⁸ and R⁹ are the same or different and each is hydrogen, alkyl, aralkyl or phenyl, and
R⁷ is hydrogen, alkyl, aralkyl, phenyl, nitro or cyano, or R⁶ and R⁷ combinedly form a heterocycle optionally having oxygen atom, sulfur atom or optionally substituted nitrogen atom additionally in the ring;
R¹ is a hydrogen, an alkyl, or a cycloalkyl, a cycloalkylalkyl, a phenyl or an aralkyl, which optionally has a substituent on a ring; or
R and R¹ combinedly form, together with the adjacent nitrogen atom, a heterocycle optionally having oxygen atom, sulfur atom or optionally substituted nitrogen atom additionally in the ring;
R² and R³ are the same or different and each is a hydrogen, an alkyl, an aralkyl, a halogen, a nitro, an amino, an alkylamino, an acylamino, a hydroxy, an alkoxy, an aralkyloxy, a cyano, an acyl, a mercapto, an alkylthio, an aralkylthio, a carboxy, an alkoxycarbonyl, a carbamoyl, an alkylcarbamoyl or an azide;
R⁴ is a hydrogen or an alkyl;
R⁵ is a heterocycle containing nitrogen, which is selected from the group consisting of pyridine, pyrimidine, pyridazine, triazine, pyrazole, triazole, pyrrolopyridine, pyrazolopyridine, imidazopyridine, pyrrolopyrimidine, pyrazolopyrimidine, imidazopyrimidine, pyrrolotriazine, pyrazolotriazine, triazolopyridine, triazolopyrimidine, cinnoline, quinazoline, quinoline, pyridopyridazine, pyridopyrazine, pyridopyrimidine, pyrimidopyrimidine, pyrazinopyrimidine, naphthylidine, tetrazolopyrimidine, thienopyridine, thienopyrimidine, thiazolopyridine, thiazolopyrimidine, oxazolopyridine, oxazolopyrimidine, furopyridine, furopyrimidine, 2,3-dihydropyrrolopyridine, 2,3-dihydropyrrolopyrimidine, 5,6,7,8-tetrahydropyrido-[2,3-d]pyrimidine, 5,6,7,8-tetrahydro-1,8-naphthylidine and 5,6,7,8-tetrahydroquinoline, provided that when said heterocycle containing nitrogen forms a hydrogenated aromatic ring, carbon atom in the ring is optionally carbonyl, and said heterocycle containing nitrogen optionally has a substituent; and
A is the formula 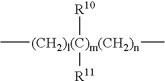
wherein R¹⁰ and R¹¹ are the same or different and each is hydrogen, alkyl, haloalkyl, aralkyl, hydroxyalkyl, carboxy or alkoxycarbonyl, or R¹⁰ and R¹¹ combinedly form cycloalkyl, and, m and n are each 0 or an integer of 1-3,
or an isomer thereof.

3. A stent for implantation in a blood vessel, wherein-the stent is coated with a compound having the structure: 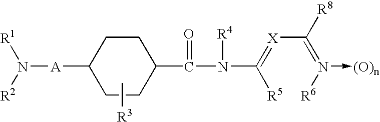
wherein:
R¹ and R² are the same or different and each is hydrogen, alkyl, or cycloalkyl, cycloalkylalkyl, phenyl, aralkyl, piperidyl or pyrrolidinyl, which may have substituent on the ring, or a group of the formula 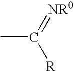
wherein
R is hydrogen, alkyl, —NR′R″ (where R′ and R″ are the same or different and each is hydrogen, alkyl, aralkyl or phenyl),
R⁰ is hydrogen, alkyl, aralkyl, phenyl, nitro or cyano, or
R and R⁰ may combinedly form a heterocyclic ring which may have, in the ring, oxygen atom, sulfur atom or optionally substituted nitrogen atom, or
R¹ and R² combinedly are alkylidene or phenylalkylidene, or R¹ and R² form, together with the nitrogen atom binding therewith, a heterocyclic ring which may have, in the ring, oxygen atom, sulfur atom or optionally substituted nitrogen atom;
R³ and R⁴ are each hydrogen or alkyl;
A is a single bond or alkylene;
X is ═C(R⁷)— or ═N—;
R⁵ and R⁶ together are a group of the formula
—CRa═CRb—,
—NRa—C(═Rb)— or
—C(═Ra)—NRb—,
wherein
Ra and Rb combinedly form an optionally hydrogenated 5- or 6-membered aromatic ring which may have, in the ring, at least one of nitrogen atom, sulfur atom and oxygen atom;
R⁷ and R⁸ are the same or different and each is hydrogen, halogen, alkyl, alkoxy, aralkyl, haloalkyl, nitro, —NReRf
{wherein Re and Rf are the same or different and each is hydrogen, alkyl, —COR⁹, —COOR^{9′}, —SO₂R^{9′} (where R⁹ is hydrogen, alkyl, phenyl or aralkyl and R^{9′} is alkyl, phenyl or aralkyl), or Re and Rf form, together with the nitrogen atom binding therewith, a heterocyclic ring which may have, in the ring, oxygen atom, sulfur atom or optionally substituted nitrogen atom}, cyano, azido, optionally substituted hydrazino, —COOR¹⁰, —CONR¹¹R¹² (wherein R¹⁰⁻¹² are each hydrogen, alkyl, phenyl or aralkyl); and
n is 0 or 1;
or an isomer thereof.

4. A stent for implantation in a blood vessel, wherein the stent is coated with a compound having the structure: 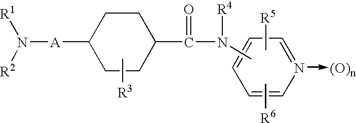
wherein
R¹ and R² are the same or different, and respectively represent:
hydrogen, C₁₋₁₀ alkyl, C₂₋₅ alkanoyl, formyl, C₁₋₄ alkoxycarbonyl, amidino, C₃₋₇ cycloalkyl, C₃₋₇ cycloalkylcarbonyl, unsubstituted or substituted phenyl, phenylalkyl, benzoyl, naphthoyl, phenylalkoxy-carbonyl, pyridylcarbonyl or piperidyl, wherein the substituent is selected from the group consisting of halogen, C₁₋₄ alkyl, C₁₋₄ alkoxy, phenylalkyl, nitro or amino,
R¹ and R² together form unsubstituted or substituted benzylidene, pyrrolidylidene or piperidylidene, wherein the substituent is selected from the group consisting of halogen, C₁₋₄ alkyl, C₁₋₄ alkoxy, phenylalkyl, nitro or amino, or
R¹ or R² together with the adjacent nitrogen atom form pyrrolidinyl, piperidino, piperazinyl, morpholino, thiomorpholino or phthalimido,
R³ represents hydrogen or C₁₋₄ alkyl,
R⁴ represents a hydrogen or C₁₋₄ alkyl,
R⁵ represents hydrogen, hydroxy, C₁₋₄ alkyl or phenylalkoxy, R⁶ represents hydrogen or C₁₋₄ alkyl,
A represents single bond, C₁₋₅ straight chain alkylene, or alkylene which is substituted by C₁₋₄ alkyl and
n represents 0 to 1,
or an isomer thereof.

5. A stent for implantation in a blood vessel, wherein the stent is coated with a compound comprising an amide compound having the structure: 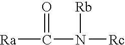
wherein
Ra is a group of the formula: 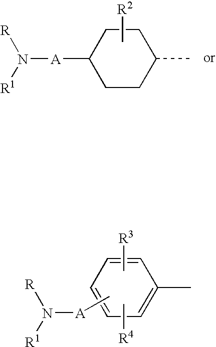
in the formulas (a) and (b),
R is hydrogen, alkyl or cycloalkyl, cycloaalkyl, phenyl or aracyl, which optionally have a substituent on the ring, or a group of the formula: 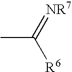
wherein R⁶ is hydrogen, alkyl or formula: —N R⁸NR⁹ wherein R⁸ and R⁹ are the same or different and each is hydrogen, alkyl, aralkyl or phenyl, R⁷ is hydrogen, alkyl, aralkyl, phenyl, nitro or cyano, or R⁶ and R⁷ in combination show a group forming a heterocycle optionally having, in the ring, oxygen atom, sulfur atom or optionally substituted nitrogen atom,
R¹ is hydrogen, alkyl or cycloalkyl, cycloalkylalkyl, phenyl or aralky, which optionally have a substituent on the ring, or
R and R¹ in combination form, together with the adjacent nitrogen atom, a group forming a heterocycle optionally having, in the ring, oxygen atom, sulfur atom or optionally substituted nitrogen atom,
R² is hydrogen or alkyl,
R³ and R⁴ are the same or different and each is hydrogen, alkyl, aralkyl, halogen, nitro, amino, alkylamino, acylamino, hydroxy, alkoxy, aralkyloxy, cyano, acyl, mercapto, alkylthio, aralkylthio, carboxy, alkoxycarbonyl, carbamoyl, alkylcarbamoyl or azide, and
A is a group of the formula: 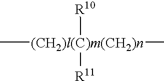
wherein R¹⁰ and R¹¹ are the same or different and each is hydrogen, alkyl, haloalkyl, aralkyl, hydroxyalkyl, carboxy or alkoxycarbonyl, or R¹⁰ and R¹¹ show a group which forms cycloalkyl in combination and l, m and n are each 0 or an integer of 1-3,
Rb is a hydrogen, an alkyl, an aralkyl, an aminoalkyl or a mono or dialkylaminoalkyl; and
Rc is an optionally substituted pyridine, triazine, pyrimidine, pyrrolopyridine, pyrazolopyridine, pyrazolopyrimidine, 2,3-dihydropyrrolopyridine, imidazopyridine, pyrrolopyrimidine, imindazopyrimidine, pyrrolotriazine, pyrazolotriazine, triazolopyridine, triazolopyrimidine, or 2,3-dihydropyrrolopyrimidine,
or an isomer thereof.

6. A stent for implantation in a blood vessel, wherein the stent is coated with a compound having the structure: 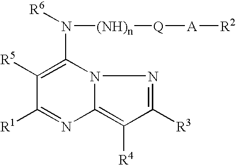
wherein
R¹ is hydrogen, lower alkyl which may have thienyl, lower alkoxy, lower alkylthio, oxo or hydroxyl as a substituent, cycloalkyl, thienyl, furyl, lower alkenyl, or R¹ phenyl, said R¹ phenyl having 1 to 3 substituents selected from the group consisting of lower alkyl, lower alkoxy, phenylthio and halogen; R² is naphthyl, cycloalkyl, furyl, thienyl, pyridyl, halogen-substituted pyridyl, phenoxy, halogen-substituted phenoxy, or phenyl which may have 1 to 3 substituents selected from the group consisting of lower alkyl, lower alkoxy, halogen, nitro, halogen-substituted lower alkyl, halogen-substituted lower alkoxy, lower alkoxycarbonyl, hydroxyl, phenyl (lower)alkoxy, amino, cyano, lower alkanoyloxy, phenyl and di(lower)alkoxyphosphoryl(lower)alkyl; R³ is hydrogen, phenyl or lower alkyl; R⁴ is hydrogen, lower alkyl, lower alkoxycarbonyl, phenyl(lower)alkyl, phenyl, phenylthio-substituted phenyl, or halogen; R⁵ is hydrogen or lower alkyl; R⁶ is hydrogen, lower alkyl, phenyl(lower)alkyl, or an R⁶ benzoyl, said R⁶ benzoyl having 1 to 3 substituents selected from the group consisting of lower alkoxy, halogen-substituted lower alkyl and halogen; R¹ and R⁵ may conjointly form lower alkylene; Q is carbonyl or sulfonyl; A is a single bond, lower alkylene or lower alkenylene; and n is 0 or 1,
or an isomer thereof.

7. A stent for implantation in a blood vessel, wherein the stent is coated with an inhibitor of Rho kinase.

8. The stent of any one of claims 1-7, wherein the stent is also coated with one or more of rapamycin, taxol, actinomycin D, heparin, C3 exoenzyme, or an inhibitor of RhoA.

9. A method of treating restenosis in a subject which comprises implanting in the subject the stent of any one of claims 1-7.

10. The method of claim 9, wherein the restenosis occurs after angioplasty or vascular stent placement.

11. The method of claim 10, wherein the restenosis occurs after coronary artery stent placement, peripheral artery stent placement, or cerebral artery stent placement.
